(19) United States
(12) Patent Application Publication
(10) Pub. No.: US 2003/0003572 A1
(43) Pub. Date: Jan. 2, 2003

(54) ISOLATION AND ENRICHMENT OF NEURAL STEM CELLS FROM UNCULTURED TISSUE BASED ON CELL-SURFACE MARKER EXPRESSION

(76) Inventors: DAVID J. ANDERSON, ALTADENA, CA (US); SEAN MORRISON, PASADENA, CA (US)

Correspondence Address: MINTZ LEVIN, ONE FINANCIAL CENTER, BOSTON, MA, 02111

(21) Appl. No.: 09/263,359

(22) Filed: Mar. 5, 1999

(51) Int. Cl. A61K 48/00; C12N 5/08

(52) U.S. Cl. 435/325; 435/368; 435/377; 435/378; 435/383; 435/395; 424/93.21

(57) ABSTRACT

The invention provides methods for the prospective identification, isolation, and self-renewal of neural stem cells from the mammalian peripheral nervous system and compositions of neural stem cells derived from uncultured tissue. Using flow-cytometry, neural crest derived cells of embryonic peripheral nerve were fractionated based on cell surface markers. The isolated p75⁺ P₀ cells from embryonic sciatic nerve were phenotypically and functionally indistinguishable from neural crest stem cells (NCSCs) previously isolated from neural tube explant cultures. Furthermore, freshly isolated p75⁺ P₀ cells gave rise to both neurons and glia when transplanted in vivo. Cell cycle analysis and BrdU labeling indicated that p75⁺ P₀NCSCs persist in the peripheral nerve by undergoing self-renewing divisions after neural crest migration has ceased.

This application claims priority under 35 U.S.C. § 119(e) to U.S. Provisional Patent Application Ser. No. 60/155,608, filed Jan. 12, 1999.

### STATEMENT REGARDING FEDERALLY SPONSORED RESEARCH

This invention was made with United States Government support under a program project grant awarded by the National Institutes of Health. The Government may have certain rights in the invention.

### TECHNICAL FIELD OF THE INVENTION

This invention relates generally to methods of neural stem cell culture, and particularly to the isolation or enrichment of neural stem cells.

### BACKGROUND OF THE INVENTION

Stem cells are self-renewing multipotent progenitors with the broadest developmental potential in a given tissue at a given time (see, Morrison et al., 88 Cell 287-298 (1997)). A great deal of interest has recently been attracted by studies of stem cells in the nervous system, not only because of their importance for understanding neural development but also for their therapeutic potential in the treatment of neurodegenerative diseases.

A limitation in the study of neural stem cells has been the inability to identify neural stem cells prospectively in vivo (see, Gage, 8 Current Opinion in Neurobiology 671-676 (1998)). Thus far, no markers have been available to isolate neural stem cells or to distinguish neural stem cells from restricted neural progenitors in vivo. Neural stem cells have so far been isolated only after a period of growth in culture, which growth could change their properties. It is therefore not yet clear whether populations of cells that exhibit multipotency and self-renewal in vitro derive from corresponding cells with similar properties in vivo. Furthermore, it is not clear whether the cells change properties during in vitro culture in ways that reduce the cells' ability to engraft and differentiate when transplanted in vivo.

The neural crest is a model system to study the biology of mammalian neural stem cells (see, Anderson et al., U.S. Pat. Nos. 5,589,376, 5,824,489, 5,654,183, 5,693,482, 5,672,499, and 5,849,553, all incorporated by reference). Neural crest stem cells can be isolated by incubating mid-gestation rat neural tube explants in culture for 24 hours. Neural crest cells migrate out of the cultured neural tubes, forming a monolayer in the culture dish. In these cultures, cells expressing the low-affinity neurotrophin receptor, p75, are a nearly pure population of neural crest stem cells (NCSCs). NCSCs are thus defined as cells that could self-renew as well as giving rise to neurons, glia, and smooth muscle in vitro. NCSCs respond to instructive lineage determination factors bone morphogenic protein (BMP2), glial growth factor (GGF), and transforming growth factor β (TGFβ) by differentiating into neurons, glia, and smooth muscle respectively (see Shah et al., 77 Cell 349-360 (1994); Shah et al., 85 Cell 331-343 (1996); Shah & Anderson, 94 Proc. Natl. Acad. Sci. U.S.A. 11369-11374 (1997), respectively).

In vivo, neural crest cells delaminate from the dorsal neural tube and migrate extensively before aggregating to form the ganglia and neuroendocrine tissues of the peripheral nervous system (PNS). Peripheral nerves contains glial (Schwann) cells which are derived from the neural crest. In rats, by E14.5, two to four days after neural crest emigration from the trunk neural tube has ceased, the sciatic nerve contains Schwann cell precursors. Over the next few days of development, these Schwann cell precursors overtly differentiate to Schwann cells. Until now, it has not been known whether these Schwann cell precursors were already committed to glial fates or still retain other developmental potentials as well. Knowledge of whether these progenitors are really lineage committed is critical to an understanding of how growth factors and transcription factors regulate peripheral nerve development.

### SUMMARY OF THE INVENTION

The invention provides methods for the prospective identification, isolation, enrichment, and self-renewal of stem cells from uncultured tissue, using cell surface markers and flow cytometry to separate stem cells from other cells. The invention also provides compositions of neural stem cells derived from uncultured neural tissue.

In one embodiment, the invention provides a method for prospectively identifying, isolating, or enriching for self-renewing multipotent neural crest stem cells (NCSCs) in vivo among populations of post-migratory neural crest cells. Previously, the lack of such a prospective isolation method has hampered the demonstration of neural stem cell self-renewal in vivo, not only for NCSCs in the peripheral nervous system (PNS), but also for neural stem cells in the central nervous system (CNS), as well.

The neural stem cells of the invention are useful for screening assays in the isolation and evaluation of factors associated with the differentiation and maturation of neural cells. The neural stem cells the isolation and evaluation of factors associated with the differentiation and maturation of cells are also useful for transplantation into subjects. In one embodiment, transplanted NCSCs can differentiate to new neurons, glia or smooth muscle. NCSCs are thus useful to repair lesions, to ameliorate neurodegenerative disease, or to engraft genetically modified cells for gene therapy. The persistence of NCSCs, demonstrated herein, is of potential therapeutic importance, and may explain the origin of some PNS tumors in humans.

### BRIEF DESCRIPTION OF THE DRAWINGS

FIG. 1A-1C is a series of fluorescence activated cell sorting (FACS) profiles of E14.5 rat sciatic nerve cells. Sciatic nerves were dissociated by either treating with trypsin and collagenase (FIG. 1A and 1C) or with hyaluronidase and collagenase (FIG. 1B). Cells are either unstained (FIG. 1A), or stained with antibodies against p75 and P₀ (FIG. 1B, 1C). At the concentrations used, none of the antibodies exhibited non-specific staining when tested by FACS on telencephalon or fetal liver cells. The phenotypically defined subsets of cells are indicated in FIG. 1B and 1C.

FIG. 2A-2F is the results of cell cycle analysis of unseparated and p75⁺ P₀⁻ sciatic nerve cells by FACS. Each cell population was stained with Hoechst 33342 to indicate DNA content and pyronin Y to indicate RNA content. In each panel the lower left quadrant contains cells in G₀ (2n DNA, low RNA content), the upper left quadrant contains cells in G₁ (2n DNA, higher RNA content), and the upper right quadrant contains cells in S, G₂, and M phases of the cell cycle (>2n DNA, high RNA content). The percentage of live cells in S/G₂/M phases is indicated. FIG. 2A shows adult rat splenocytes, a quiescent control. FIG. 2B shows E14.5 rat telencephalon cells, a rapidly cycling control population. FIG. 2C and 2D show unseparated E14.5 rat sciatic nerve cells from two different rats. FIG. 2E and 2F show p75⁺ P₀⁻ sciatic nerve cells from the same two rats.

### DETAILED DESCRIPTION OF THE INVENTION

Introduction. The invention both extends the previous stem cell art and provides fundamental new advances of importance to the entire field of nervous system stem cell biology.

Using antibodies against the cell surface markers, one skilled in the art can fractionate neural crest-derived cells of the embryonic peripheral nerve by flow cytometry. These fractionated neural crest-derived cells are phenotypically and functionally indistinguishable from neural crest stem cells (NCSCs). The method provides for the isolation or enrichment for NCSCs directly from uncultured tissue, without extensive culture in vitro. Previously, NCSCs have been isolated from neural crest cells that migrate out of explant cultures of mid gestation neural tube.

In one embodiment, these p75⁺ P₀⁻ cells are fractionated from other cells of the embryonic peripheral nerve, such as sciatic nerve, to provide a population of cells enriched in NCSCs. After isolation, these multipotent and self-renewing post-migratory neural crest cells can be cultured in vitro, or they can be transplanted directly in vivo without ever being cultured. In vivo, these cells exhibit stem cell properties including self-renewal and multilineage differentiation. Freshly isolated p75⁺ P₀⁻ cells gave rise to both neurons and glia after direct transplantation into chick embryos, demonstrating that the neuronal potential of these cells is not a culture artifact. Finally, in vivo cell cycle analysis and bromodeoxyuridine (BrdU) labeling indicates that p75⁺P₀⁻NCSCs persist in the peripheral nerve by undergoing self-renewing divisions after neural crest migration has ceased. Stem cells are thus distinguishable from other cell types in the PNS by surface marker expression. Taken together, these data show that multipotent neural crest cells self-renew in vivo and persist into late gestation, at least a week after the onset of neural crest migration in rats (and for the equivalent gestational time period in other animals and humans).

The invention provides, for the first time, a method whereby any nervous system stem cell can be isolated from uncultured tissue based on cell-surface marker expression. The invention thus provides an important methodological innovation, the use of monoclonal antibodies to a cell surface marker to enrich for, isolate, and identify stem cells from uncultured tissue, a method extensible to other neural stem cell populations as well. Many of the applications used for hematopoietic stem cells, including transplantation and gene therapy, are thus applicable to neural stem cells. More specifically, the invention facilitates the isolation of NCSCs by greatly expanding the sources from which these NCSCs can be isolated. Now, it is not necessary to isolate these cells from the neural tube at mid-gestation, since NCSCs can be obtained from the sciatic nerve into late gestation. The invention for the first time allows the manipulation of neural stem cells with the same facility as hematopoietic stem cells.

One of skill in the art can isolate or enrich for stem cells from uncultured tissue, such as dissociated nerve. Nerve tissue can be enzymatically dissociated. For, example, E14 sciatic nerve can be dissociated using a combination of enzymes, such as hyaluronidase and collagenase (see, EXAMPLE 1). Other combinations of enzymes can be also be used, e.g., trypsin. E14 sciatic nerve can also be dissociated nonenzymatically, by trituration or other mechanical dissociation techniques. At later stages of fetal development (e.g., E17 and older sciatic nerve), enzymatic dissociation is probably required. The choice of surface markers used to isolate and the ability to enrich for stem cells by flow cytometry depends on the tissue dissociation method, because methods that include proteases can cause loss of some protein and protein associated cell surface antigens.

Stem cells. The term “stem cell” means (1) that the cell is an undifferentiated cell capable of generating one or more kinds of differentiated derivatives; (2) that the cell has extensive proliferative capacity; and (3) that the cell is capable of self-renewal or self-maintenance (see, Potten et al., 110 Development 1001 (1990)). The term “neural crest stem cell” (NCSC) refers to a cell derived from the neural crest which is characterized by having the properties (1) of self-renewal and (2) asymmetrical division; that is, one cell divides to produce two different daughter cells with one being self (renewal) and the other being a cell having a more restricted developmental potential, as compared to the parental neural crest stem cell (see, Anderson et al., U.S. Pat. Nos. 5,589,376, 5,824,489, 5,654,183, 5,693,482, 5,672,499, and 5,849,553, all incorporated by reference). This does not mean, however, that each and every cell division of a neural crest stem cell gives rise to an asymmetrical division (see, EXAMPLE 2, below). A division of a neural crest stem cell can also result only in self-renewal, in the production of more developmentally restricted progeny only, or in the production of a self-renewed stem cell and a cell having restricted developmental potential.

The term “multipotent neural stem cell” refers to a cell having properties similar to that of a neural crest stem cell, but which is not necessarily derived from the neural crest. Rather, such multipotent neural stem cells can be derived from various other tissues including neural epithelial tissue from the brain or spinal cord of the adult or embryonic central nervous system (CNS) or neural epithelial tissue which may be present in tissues comprising the PNS. In addition, multipotent neural stem cells may be derived from other tissues such as lung, bone and the like utilizing the methods disclosed herein. Such cells are capable of regeneration and differentiation to different types of neurons or glia, e.g., PNS and CNS neurons and glia, to smooth muscle cells, when the neural stem cells are NCSCs, or to neuronal or glial progenitors thereof Thus, the neural crest stem cells (NCSCs) described above are at least multipotent in that they are capable, under the conditions described, of self-regeneration and differentiation to neurons, glia and smooth muscle in vitro. Thus, a NCSC is a multipotent neural stem cell derived from a specific tissue, i.e., the embryonic neural tube.

Neural stem cells, including NCSCs, can be operationally characterized by cell surface markers. These cell surface markers can be bound by reagents that specifically bind to the cell surface markers. For example, proteins or carbohydrates on the surfaces of neural stem cells can be immunologically recognized by antibodies specific for the particular protein or carbohydrate. The set of markers present on the cell surfaces of neural stem cells is characteristic for neural stem cells. Therefore, neural stem cells can be selected by positive and negative selection of cell surface markers. A reagent that binds to a neural stem cell “positive marker” (i.e., a marker present on the cell surfaces of neural stem cells) can be used for positive selection of neural stem cells. A reagent that binds to a neural stem cell “negative marker” (i.e., a marker not present on the cell surfaces of neural stem cells) can be used for the negative selection of those cells in the population that are not neural stem cells (i.e., for the elimination of cells that are not neural stem cells). A “combination of reagents” is at least two reagents that bind to cell surface markers either present (positive marker) or not present (negative marker) on the surfaces of neural stem cells, or to a combination of positive and negative markers (for example, p75 and P₀).

Neural stem cell positive markers may also be found on other cells derived from neural stem cells, e.g., glial and neuronal progenitor cells of the PNS and CNS, in addition to being found on neural stem cells. An example is the cell surface expression of p75, the low-affinity nerve growth factor receptor (LNGFR) found on neural crest stem cells of n rat, humans, and monkeys. p75 is found on several mammalian and bird cell types including neural crest cells and Schwann cells (glial cells of the PNS) as well as on the surface of cells in the embryonic CNS (see, e.g., Yan et al., 8 J. Neurosci. 3481-3496 (1988) (rat); Heuer et al., 5 Neuron 283-296 (1980) (chick)) Antibodies specific for p75 have been identified for p75 from rat (217c; see, Peng et al., 215 Science 1102-1104 (1982); 192-Ig; see, Brockes et al., 266 Nature 364-366 (1977)) and human (Ross et al. 81 Proc. Natl. Acad. Sci. U.S.A. 6681-6685 (1984)). The monoclonal antibody against human p75 cross-reacts with p75 from monkeys. Using the techniques known to those of skill in the art, monoclonal antibodies specific for p75 from any desired species can be generated (see, Harlow et al., Antibodies: A Laboratory Manual (Cold Spring Harbor Laboratory Press, 1988). It is not always necessary to generate polyclonal- or monoclonal antibodies that are species specific. Monoclonal antibodies against an antigenic determinant from one species may react against that antigen from more than one species. For example, as stated above, the antibody directed against the human p75 also recognizes p75 on monkey cells.

For negative selection, NCSCs can be isolated or enriched for by the absence of cell surface markers associated with mature PNS neuronal or glial cells. These markers include the myelin protein P₀ in PNS glial cells. P₀ is a peripheral myelin protein that is expressed by committed Schwann cells. P₀ also is expressed at relatively low levels in a subset of migrating neural crest cells in both birds and mammals. P₀ expression during and shortly after migration to the early sciatic nerve has been interpreted as reflecting an early commitment of neural crest cells to a glial fate (see, Lee et al., 8 Molecular and Cellular Neuroscience 336-350 (1997)). However, some NCSCs (as well as M-only progenitors) are present in the p75⁺P₀⁺fraction (see, TABLE 3, and 99% of p75^{-/low} P₀⁺ cells give rise to M-only colonies (see, TABLE 3). These results indicates that expression of P₀ does not necessarily signify commitment to a glial fate. Furthermore, neural stem cells, including NCSCs, can express detectable levels of P₀ mRNA, and possibly low levels of P₀ protein, without being detectably identified as expressing the P₀ protein marker or being selected as P₀⁺ by flow cytometry.

In one embodiment, the “combination of reagents” is an antibody to p75 and an antibody to P₀.

The use of antibodies specific for neural stem cell surface markers results in the method of the invention being useful for the isolation or enrichment of multipotent neural stem cells from tissues other than embryonic neural tubes. For example, p75 is expressed in cells of the embryonic CNS of the rat and chick. Other mammalian and bird species have a similar pattern of p75 expression; studies in human by Loy, et al. 27 J. Neurosci Res. 651-654 (1990) with monoclonal antibodies against the human p75 are consistent with this expectation. Thus, the method of the invention is useful for the enrichment or isolation of human neural stem cells. Also, the finding that NCSCs persist later than expected during fetal development indicates that NCSCs exist in peripheral nerves postnatally. Small numbers of neurons have been reported to emerge from explants of postnatal sciatic nerves (Barakat-Walter, 161 Developmental Biology 263-273 (1994)). Furthermore, since neural stem cells are also found in the CNS (see, Weiss et al., U.S. Pat. Nos. 5,750,376 and 5,851,832, Johe, U.S. Pat. No. 5,753,506, all incorporated herein by reference), antibodies to neural cell-specific surface markers are useful in isolating multipotent neural stem cells from the CNS, PNS, and from other tissue sources. Methods using antibodies to neural cell-specific surface markers are useful in isolating multipotent neuroepithelial stem cells from the nerve tissue sources (see, Rao et al., PCT/US98/093630; Rao et al., 95(7) Proc. Natl. Acad. Sci. U.S.A. 3996-4001 (1998), both incorporated by reference).

The term “neuronal progenitor cell” refers to a cell which is intermediate between the fully differentiated neuronal cell and a precursor multipotent neural stem cell from which the fully differentiated neuronal cell develops. The term “PNS neuronal progenitor cell” means a cell which has differentiated from a mammalian neural crest stem cell which is committed to one or more PNS neuronal lineages and is a dividing cell but does not yet express surface or intracellular markers found on more differentiated, non-dividing PNS neuronal cells. Such progenitor cells are preferably obtained from neural crest stem cells isolated from the embryonic neural crest which have undergone further differentiation. However, equivalent cells may be derived from other tissue. When PNS neuronal progenitor cells are placed in appropriate culture conditions they differentiate into mature PNS neurons expressing the appropriate differentiation markers, for example, peripherin, neurofilament and high-polysialic acid neural cell adhesion molecule (high PSA-NCAM).

Cultures. The invention also provides compositions of multipotent neural stem cell cultures. These cell cultures could not have been provided without the development of the isolation methods of the invention. The invention provides NCSC compositions, which can serve as a source for neural crest cell derivatives such as neuronal and glial progenitors of the PNS. In turn, the neuronal and glial progenitors of the PNS are a source of PNS neurons and glia. The invention also provides CNS neural stem cell compositions in which the stem cells are prepared from uncultured tissue (compare, Weiss et al., U.S. Pat. Nos. 5,750,376 and 5,851,832, both incorporated herein by reference). The invention also provides neuroepithelial stem cell compositions in which the neuroepithelial stem cells are prepared from uncultured tissue (compare, Rao et al., PCT/US98/093630; Rao et al, 95(7) Proc. Natl. Acad. Sci. U.S.A. 3996-4001, 1998), both incorporated by reference).

The culture medium can be a chemically defined medium which is supplemented with chick embryo extract (CEE) as a source of mitogens and survival factors to allow the growth and self renewal of rat neural crest stem cells (see, EXAMPLE 1, below). Other serum-free culture medium containing one or more predetermined growth factors effective for inducing multipotent neural stem cell proliferation known to those of skill in the art can be used to isolate and propagate neural crest stem cells from other bird and mammalian species, such as human (see, Weiss et al., U.S. Pat. Nos. 5,750,376 and 5,851,832; Johe, U.S. Pat. No. 5,753,506; Atlas et al., Handbook of Microbiological Media (CRC Press, Boca, Raton, La., 1993); Freshney, Cutler on Animal Cells, A Manual of Basic Technique, 3d Edition (Wiley-Liss, New York, 1994), all incorporated herein by reference).

The culture medium for the proliferation of neural stem cells thus supports the growth of neural stem cells and the proliferated progeny. The “proliferated progeny” are undifferentiated neural cells, including neural stem cells, since neural stem cells have a self-renewal capability in culture (see, EXAMPLE 2). In vitro cell culture compositions of the invention can contain a high percentage of self-renewing multipotent neural stem cells, preferably at least 50%, more preferably 60% (as described in TABLE 3). In vitro cell culture compositions of the invention can also contain a high percentage of cells having cell surface markers characteristic of neural stem cells. In one embodiment, the cell cultures contain at least 80% p75⁺ cells.

For NCSC compositions, the culture medium may contain instructive factors, such as growth factors from the TGF-β superfamily. The term “instructive factor” refers to one or more factors that can cause the differentiation of neural stem cells primarily to a single lineage, e.g., glial, neuronal or smooth muscle cell. Thus, a factor which is instructive for smooth muscle cell differentiation is one which causes differentiation of neural stem cells to smooth muscle cells at the expense of the differentiation of such stem cells into other lineages such as glial or neuronal cells. Having identified that mammalian serum contains one or more instructive factors for smooth muscle cell differentiation, such instructive factors can be identified by fractionating mammalian serum and adding back one or more such fractions to a neural stem cell culture to identify one or more fractions containing instructive factors for smooth muscle cell differentiation. Positive fractions can then be further fractionated and reassayed until the one or more components required for instructive differentiation to smooth muscle cells are identified.

The term “growth factors from the TGF-β superfamily” means growth factors related to transforming growth factor beta- 1 (“TGF-β1”). Such TGF-β superfamily growth factors may or may not exert a similar biological effect to TGF-β1, the prototypic member of the TGF-β superfamily. By way of example, members of the TGF-β superfamily of growth factors include but are not limited to naturally occurring analogues (e.g. TGF-β2, -β3, -β4), and any known synthetic or natural analogues of TGF-β1 in addition to related growth factors exemplified by bone morphogenic proteins 2 and 4 (“BMP-2” and “BMP-4”). These compounds can be purified from natural sources or may be produced by recombinant DNA techniques and may or may not be substantially pure. Variants and fragments retaining the property of causing differentiation are included in the definition of the members of this superfamily. Furthermore, the term bone morphogenic protein (“BMP”) refers to a group of growth factors which are members of the TGF-β superfamily. The growth factors described herein can be administered individually or in combination with each other.

The instructive factor may additionally or alternatively be an NRG-1. NRG-1 is expressed on motor axons in the nerve, and is genetically essential for proper Schwann cell development. NRG-1 (also known as glial growth factor) promotes glial differentiation by NCSCs in an instructive manner (Shah et al., 77 Cell 349-360 (1994)), and can cause a rapid loss of neurogenic capacity by NCSCs in the absence of cell death (Shah & Anderson, 94 Proc. Natl. Acad. Sci. U.S.A. 11369-11374 (1997)). Neuregulin also promotes the survival and proliferation of Schwann cells and their progenitors.

As demonstrated in EXAMPLE 3 below, NRG-1 acts instructively on NCSCs isolated from sciatic nerve. NRG- 1 also promoted the survival of all neural progenitors (see, plating efficiencies, TABLES 3 and 5) as well as the proliferation of -Schwann (S-only) and myofibroblast (M-only) progenitors within the sciatic nerve. These effects were independent and could clearly be distinguished from each other; the promotion of survival could not explain the instructive effect and vice versa. Thus, there is no conflict between these different neuregulin functions. Taken together, these data are consistent with the idea that NRG- 1 in the peripheral nerve plays multiple roles in Schwann cell development including the restriction of NCSCs to non-neurogenic fates.

Enrichment of neural stem cells and plating efficiency. The plating efficiency of the p75⁺P₀⁻ cells was around 25% under both standard conditions (TABLE 1), and in cultures supplemented with BMP2. While this calculation compares favorably with previous clonal analyses of multipotent neural progenitors (Reynolds et al., 12 J. Neurosci. 4565-4574 (1992); Kilpatrick & Bartlett, 10 Neuron 255-265 (1993); Gritti et al., 16 J. Neurosci. 1091-1100 (1996); Johe et al., 10 Genes & Dev. 3129-3140 (1996)), it raises the question of whether other progenitor types that do not form colonies under these culture conditions may be concealed within the p75⁺P₀⁻population. This possibility is unlikely given that all other progenitor types within the sciatic nerve formed colonies under our culture conditions. Indeed, these other progenitor populations had higher plating efficiencies than the p75⁺P₀⁻cells (TABLE 3).

Lineage restriction in the peripheral nerve is more complex and dynamic than previously anticipated. The results presented here show that PNS development is surprisingly more dynamic and plastic than previously thought.

The PNS was thought to form relatively quickly during early to mid-gestation, with neural crest progenitors differentiating rapidly after migrating. Multipotent progenitors, such as those in embryonic neural tube cells and in early migrating neural crest cells, were thought to become restricted very quickly during migration and not persist for very long among post-migratory neural crest cells. Investigational approaches used in birds had suggested that NCSCs differentiate relatively quickly, such that within a few days after migration all cell fates are determined within the PNS.

Neural crest derived cells in the E14 sciatic nerve were previously thought to be Schwann cell precursors, fated to differentiate into Schwann cells. The developmental potential of these Schwann precursors was thought to be different from neural crest progenitors, because p75⁺ cells from the sciatic nerve were observed to stain with an antibody against GAP-43 while neural crest outgrowth did not. We have tested monoclonal and polyclonal anti-GAP-43 antibodies under a variety of fixation and culture conditions, but failed to observe staining other than in neurons that differentiated in NCSC colonies.

Whether or not the sciatic nerve cells express GAP-43, E14.5 sciatic nerve cells comprise a heterogeneous collection of progenitors with respect to marker expression and developmental potential, a significant proportion are phenotypically and functionally indistinguishable from NCSCs while a relatively small proportion appeared committed to the Schwann cell fate. The types of progenitors cultured from the E145 sciatic nerve show that NCSCs can generate both myofibroblast derivatives and Schwann cells in the peripheral nerve. Myofibroblast derivatives may include perineurium, epineurium, and vascular smooth muscle.

In the same way, Mac-1, a marker of mature myeloid cells, has been shown to be expressed on fetal hematopoietic stem cells (Morrison et al., Proc. Natl. Acad. Sci. U.S.A. 92, 10302-10306 (1995)). Such phenomena are consistent with the idea that the multipotency of stem cells may be reflected at the molecular level in the low-level transcription of genes whose products ultimately define different stem cell derivatives.

Transplantation of Neural Stem Cells. The neural stem cell cultures of the invention can be produced and transplanted into hosts (see, EXAMPLE 6). The method of transplantation can therefore be used for transplantation into a variety of hosts, preferably human patients. Neural stem cells transplanted into human patients is useful for the treatment of various disorders, in the PNS, in the CNS, and systemically. The ability to isolate neural stem cells from peripheral nerve biopsies could have important therapeutic applications, because NCSCs could be transplanted to the site of neural injuries, especially if the environment of the adult nerve remains permissive for NCSC survival, self-renewal, and differentiation.

Cells are delivered to the subject by any suitable means known in the art. When delivered to the CNS, then the cells are administered to a particular region using any method which maintains the integrity of surrounding areas of the brain, preferably by injection cannula. Injection methods exemplified by those used by Duncan et al., 17 J. Neurocytology 351-361 (1988), and scaled up and modified for use in humans are preferred. Methods for the injection of cell suspensions such as fibroblasts into the CNS may also be employed for injection of neural precursor cells. Additional approaches and methods may be found in Neural Grafting in the Mammalian CNS, Bjorklund & Stenevi, eds. (1985).

The neural stem cell cultures of the invention can be produced and transplanted using the above procedures to treat various neurodegenerative disorders. Such CNS disorders encompass numerous afflictions such as neurodegenerative diseases (e.g. Alzheimer's and Parkinson's), acute brain injury (e.g. stroke, head injury, cerebral palsy) and a large number of CNS dysfunctions (e.g. depression, epilepsy, and schizophrenia). In recent years neurodegenerative disease has become an important concern due to the expanding elderly population which is at greatest risk for these disorders. These diseases, which include Alzheimer's Disease, Multiple Sclerosis (MS), Huntington's Disease, Amyotrophic Lateral Sclerosis, and Parkinson's Disease, have been linked to the degeneration of neural cells in particular locations of the CNS, leading to the inability of these cells or the brain region to carry out their intended function. By providing for maturation, proliferation and differentiation into one or more selected lineages through specific different growth factors the progenitor cells may be used as a source of committed cells. In one series of embodiments, collagenase-treated neural stem cell cultures can be produced and transplanted using the above procedures for the treatment of demyelination diseases. Any suitable method for the implantation of cells near to the demyelinated targets may be used so that the cells can become associated with the demyelinated axons.

Neural stem cell cultures made according to the present invention may also be used to produce a variety of blood cell types, including myeloid and lymphoid cells, as well as early hematopoietic cells (see, Bjornson et al., 283 Science 534 (1999), incorporated herein by reference).

Methods for screening the effect of agents on neural stem cells. The neural stem cell cultures of the invention, cultured in vitro, can be used for the screening of potential neurologically therapeutic compositions, for the isolation and evaluation of factors in the compositions associated with the differentiation and maturation of cells. These compositions can be applied to cells in culture at varying dosages, and the response of the cells monitored for various time periods. Physical characteristics of the cells can be analyzed by observing cell and neurite growth with microscopy. The induction of expression of new or increased levels of proteins such as enzymes, receptors and other cell surface molecules, or of neurotransmitters, amino acids, neuropeptides and biogenic amines can be analyzed with any technique known in the art which can identify the alteration of the level of such molecules. These techniques include immunohistochemistry using antibodies against such molecules, or biochemical analysis. Such biochemical analysis includes protein assays, enzymatic assays, receptor binding assays, enzyme-linked immunosorbant assays (ELISA), electrophoretic analysis, analysis with high performance liquid chromatography (HPLC), Western blots, and radioimmune assays (RIA). Nucleic acid analysis such as Northern blots can be used to examine the levels of mRNA coding for these molecules, or for enzymes which synthesize these molecules. Alternatively, cells treated with these pharmaceutical compositions can be transplanted into an animal, and their survival, ability to form neuronal connections, and biochemical and immunological characteristics examined as previously described.

The neural stem cell cultures of the invention can be used in methods of determining the effect of a biological agents on neural cells. The term “biological agent” refers to any agent, such as a virus, protein, peptide, amino acid, lipid, carbohydrate, nucleic acid, nucleotide, drug, pro-drug or other substance that may have an effect on neural cells whether such effect is harmful, beneficial, or otherwise. Biological agents that are beneficial to neural cells are referred to herein as “neurological agents”, a term which encompasses any biologically or pharmaceutically active substance that may prove potentially useful for the proliferation, differentiation or functioning of CNS cells or treatment of neurological disease or disorder. To determine the effect of a potential biological agent on neural cells, a culture of collagenase-treated neural stem cell cultures is obtained and proliferated in vitro in the presence of a proliferation-inducing growth factor. Generally, the biological agent will be solubilized and added to the culture medium at varying concentrations to determine the effect of the agent at each dose. The culture medium may be replenished with the biological agent every couple of days in amounts, so as to keep the concentration of the agent somewhat constant.

Thus, it is possible to screen for biological agents that increase the proliferative ability of progenitor cells which would be useful for generating large numbers of cells for transplantation purposes. It is also possible to screen for biological agents which inhibit precursor cell proliferation, using collagenase-treated neural stem cell cultures. Also, the ability of various biological agents to increase, decrease or modify in some other way the number and nature of differentiated neural cells can be screened on collagenase-treated neural stem cell cultures that have been induced to differentiate. The effects of a biological agent or combination of biological agents on the differentiation and survival of differentiated neural cells can then be determined. It is also possible to determine the effects of the biological agents on the differentiation process by applying them to the neural stem cell cultures prior to differentiation.

Sciatic nerve stem cells and the origins of PNS tumors. The present discovery of the persistence of NCSCs provides an important insight into the etiology of certain PNS cancers. Peripheral neuroectodermal tumors and Ewings sarcomas often contain primitive cells with the potential to differentiate into several different neuronal and mesectodermal lineages. While these tumors may be associated with the transformation of neural crest progenitors, this association was mysterious, given the expectation that neural crest progenitors terminally differentiate early in fetal development. The present discovery of the persistence of NCSCs indicates that Ewings' sarcomas, which occur predominantly in the bones of children, may derive from the immortalization of NCSCs present in the peripheral nerve fibers that innervate the periosteum. Similarly, neurofibromas containing cells with Schwann and myofibroblast properties occur in the peripheral nerves of children, and might also derive from the transformation of NCSCs (or of S+M progenitors) during late fetal or postnatal development. The finding that NCSCs persist in rodent peripheral nerve may therefore be important for the diagnosis and treatment of PNS diseases.

The following examples are presented to more fully illustrate the preferred embodiments of the invention. These examples should in no way be construed as limiting the scope of the invention, as defined by the appended claims.

### EXAMPLE 1

### THE FETAL SCIATIC NERVE CONTAINS MULTIPOTENT AND COMMITTED NEURAL PROGENITORS

Introduction. To show that the sciatic nerve contains uncommitted neural progenitors, we dissociated E14.5-E17.5 rat sciatic nerves, and plated single cells in culture at clonal density. The individual isolated cells self-renewed and gave rise to clones containing neurons, glia, and smooth muscle-like myofibroblasts.

Preparation of sciatic nerve cell cultures. Pregnant Sprague-Dawley rats were obtained from Simonsen (Gilroy, Calif.). For timed pregnancies, animals were put together in the afternoon and the morning on which the plug was observed was designated E0.5. Sciatic nerves were dissected into ice cold Ca⁺⁺, Mg⁺⁺-free Hank's Balanced Salt Solution (HBSS) (Gibco, Grand Island, N.Y.) with 10 mM HEPES, pH 7.4 (Gibco). Nerves were pelleted by centrifuging at 450×g for three (min) at room temperature. E14-E17 nerves were usually dissociated by incubating for four minutes (min) at 37° C. in 0.025% trypsin (Gibco product 25300-054, diluted 1:1 in Ca⁺⁺, Mg⁺⁺-free HBSS) plus 1 mg/mL type 3 collagenase (Worthington, N.J.). In some tests, nerves were dissociated by incubating for 10 min at 37° C. in 1.2 mg/mL hyaluronidase (Sigma, St. Louis, product H-3884) plus 2 mg/mL type 3 collagenase. After the incubation period, digestion was quenched with 2 volumes of staining medium: L15 medium containing 1 mg/mL BSA (Gibco product 11019-023), 10 mM HEPES (pH 7.4), penicillin/streptomycin (BioWhittaker, Maryland), and 25 Ug/mL deoxyribonuclease (DNase) type 1 (Sigma, D-4527). After centrifuging, nerve cells were triturated and resuspended in staining medium. Dissociated cells were always maintained in staining medium, sometimes without DNase.

Culture of sciatic nerve cells. To arrive at conditions that promoted high plating efficiencies, and in which neuronal, glial, and mesectodermal differentiation could occur, dissociation and culture conditions were optimized. Under the standard conditions, clonal cultures were allowed to develop for 14 days, and then fixed and analyzed with immunocytochemical markers.

Sciatic nerve progenitors were typically cultured in 6-well plates (Corning, Corning N.Y.) at clonal density (fewer than 30 clones/well for 14 day cultures, or 60 clones/well for 1 to 4 day cultures). Plates were coated with poly-d-lysine (PDL) (Biomedical Technologies, Stoughton Ma.) by pipetting 50 μg/mL PDL in water onto and then off of plates within 2 min. After drying, the plates were washed with sterile distilled water (BioWhittaker) and dried again. Then plates were coated with 0.15 mg/mL human fibronectin (Biomedical Technologies) dissolved overnight in D-PBS (BioWhittaker). A series of tests was undertaken to optimize the culture medium composition. High plating efficiencies and good colony growth were consistently obtained with rat cells in the following medium: DMEM-low (Gibco product 11885-084) with 15% chick embryo extract, prepared as described by Stemple & Anderson, 71 Cell 973-985 (1992) (see also, Anderson et al., U.S. Pat. Nos. 5,589,376, 5,824,489, 5,654,183, 5,693,482, 5,672,499, and 5,849,553, all incorporated by reference), 20 ng/mL recombinant human bFGF (R&D Systems, Minn.), N2 supplement (Gibco), B27 supplement (Gibco), 50 μM 2-mercaptoethanol, 35 mg/mL (110 nM) retinoic acid (Sigma), and penicillin/streptomycin (BioWhittaker). This medium composition is described as the standard medium. Under standard conditions, cells were cultured for 6 days in standard medium, then switched to a similar medium (with 1% chick embryo extract (CEE) and 10 ng/mL bFGF) that favors differentiation for another eight days before immunohistochemical analysis of colony composition. Cultures were maintained in humidified incubators with 6 to 8% CO₂.

Immunohistochemistry. For BrdU staining, cells were fixed as described by Raff et al., 333 Nature 562-565 (1988)), blocked for 15 min in PGN (4% goat serum, 0.5% BSA, 0.1% NP-40 [Igepal, Sigma], 0.05% sodium azide in D-PBS) at room temperature, then incubated in a 1/100 dilution of anti-BrdU antibody (IU-4, Caltag) in PGN for 45 min. After washing with PGN, cells were incubated for 25 min with an anti-mouse IgG antibody conjugated to horse radish peroxidase (HRP) (Chemicon, Temecula Calif.). The histochemical reaction to detect HRP was performed using diaminobenzidine and nickelous sulfate as substrates. BrdU staining was always nuclear, and there was no background from either primary or secondary antibody in negative control cells.

In most tests in which the cellular composition of colonies was evaluated, cultures were fixed in acid ethanol (5% glacial acetic acid in 100% ethanol) for 20 min at −20° C. Plates were rinsed in PBS followed by twice in PGN. Cultures were blocked by incubating for 15 min in PGN. Peripherin was stained first by incubating for 30 min in 1/1000 anti-peripherin antibody (Chemicon) in PGN. After washing three times with PGN, peripherin staining was developed by incubating in an anti-rabbit antibody conjugated to HRP (Vector, Burlingame Calif.), followed by nickel-DAB staining. Cultures were next incubated in a mixture of 1/200 anti-GFAP (Sigma, G-3893) and 1/200 anti-SMA (Sigma, A-2S47) in PGN for 45 min at room temperature. After washing, cultures were incubated in 1/200 dilutions of anti-mouse IgG₁-phycoerythrin and anti-mouse IgG₂ₐ-FITC (Southern Biotechnology Associates) in PGN for 25 min at room temperature. After washing, nuclei were sometimes stained by incubating in 10 μg/mL DAPI in PGN for 10 min.

When cultures were stained with combinations of antibodies that did not include anti-GFAP or anti-BrdU, the cultures were usually fixed in 4% paraformaldehyde for 10 min at room temperature. MASH-1 staining was performed as described by Shah et al., 77 Cell 349-360 (1994).

Assay for developmental potentials of PNS cells. To assay developmental potentials, cells were challenged by adding to the cultures growth factors that induce differentiation. To assay for neuronal potential, cells were challenged by adding 50 ng/mL (1.6 nM) recombinant human BMP2 (Genetics Institute) to standard cultures. This is a saturating dose in terms of instructing neuronal differentiation in NCSCs (see, Shah et al., 85 Cell 331-343 (1996)). BMP2 challenged cells were incubated for 1 to 4 days before immunohistochemical analysis. To assay for glial potential, cultures were challenged by adding 50 ng/mL (1 nM) recombinant human NRG-1 (Cambridge Neurosciences). This is a saturating dose of NRG-1 with respect to instructing glial differentiation in NCSCs (see, Shah & Anderson, 94 Proc. Natl. Acad. Sci. USA 11369-11374 (1997)). Cells were cultured in the presence of NRG-1 for 14 days before analysis.

Results. Three cell types are present in these cultures: (1) neurons; (2) Schwann cells, (3) and smooth muscle-like myofibroblasts. Neurons were identified by expression of peripherin. Glial cells were identified by expression of GFAP, p75 and cytoplasmic S100β. Glial cells did not express peripherin or alpha smooth muscle actin (SMA).

Myofibroblasts were identified by co-expression of SMA and calponin. Although similar NCSC-derived cells were previously referred to as smooth muscle cells by Shah et al., 85 Cell 331-343 (1996)), the sciatic nerve-derived cells did not express the smooth muscle markers desmin or myosin light chain kinase and therefore their overall marker profile was more consistent with a related cell type that have been described as myofibroblasts by Sappino et al., 63 Laboratory Investigation 144-161 (1990). The myofibroblasts did not express the neural markers GFAP, peripherin, and p75, but did express vimentin and S100β. Although S100β has been used as a marker of Schwann cell differentiation, our observation of S100β in both glia and myofibroblasts is consistent with its widespread expression in non-neural cell types, including smooth muscle and myoepithelial cells (see, Haimoto et al., 57 Laboratory Investigation 489-498 (1987).

By triple-labeling with antibodies to peripherin, GFAP, and SMA, we identified five types of colonies-in clonal cultures of sciatic nerves from different ages (TABLE 1)

TABLE 1
The frequencies of different progenitor types from dissociated E14.5-E17.5 sciatic nerve
preparations based on the types of colonies that form in clonal culture

| Sciatic | Plating | Frequency of colony types (% ±_std. dev) | | | | |
|---|---|---|---|---|---|---|
| nerve | efficiency | N + S + M | N + S | S + M | S only | M only |
| E14.5 | 63.7 ± 16.6 | 15.8 ± 7.0^{a} | 0.4 ± 1.0 | 10.9 ± 11.9 | 19.2 ± 10.2^{a} | 53.6 ± 20.2 |
| E15.5 | 51.6 ± 13.8 | 6.8 ± 3.1^{b} | 2.4 ± 3.3 | 6.8 ± 5.1 | 31.9 ± 14.0^{ab} | 52.1 ± 22.5 |
| E16.5 | 52.0 ± 18.1 | 0.7 ± 1.4 c | 0.0 ± 0.0 | 9.0 ± 4.1 | 36.6 ± 4.9^{b} | 53.7 ± 5.6 |
| E17.5 | 52.2 ± 21.5 | 1.7 ± 2.0 c | 0.5 ± 1.0 | 9.8 ± 6.5 | 42.1 ± 14.3^{b} | 45.8 ± 12.4 |
| N, S, and M indicate the presence of neurons, Schwann cells, and myofibroblasts respectively in colonies. For example, N + S + M colonies contain neurons, Schwann cells and myofibroblasts. Plating efficiency expresses the percentage of cells added to culture that went on to form colonies analyzed after two weeks of culture. Statistics within columns of colony-type data were compared by analyses of variance followed by post-hoc T-tests. Columns containing significantly different | | | | | | |
| # statistics (p < 0.05 by anova) include letters to designate the pair-wise differences. Significantly different statistics are not followed by the same letter (e.g., a is different from b but not from a,b). | | | | | | |

A substantial number of colonies contained neurons, Schwann cells, and myofibroblasts (N+S+M). These were the largest colonies observed, containing on average 1.07±0.33×f10⁵ cells (mean±std. dev.) after 14 days of culture (corresponding to approximately 16-17 doublings). These multipotent progenitors represented almost 16% of colonies at E14.5, but their frequency declined significantly with each day of development, such that multipotent progenitors represented less than 2% of progenitors from the E17.5 sciatic nerve (TABLE 1). In a minority of tests, some infrequent colonies contained only neurons and Schwann cells (N+S). these N+S colonies were very large.

More frequent were colonies that contained no neurons, but Schwann cells and myofibroblasts (S+M). They represented up to 10% of colonies, irrespective of the stage of development between E14.5 and E17.5 (TABLE 1). These S+M colonies were intermediate in size, typically containing thousands of cells.

The sciatic nerve also contained progenitors that gave rise to only a single cell type. As expected, a substantial number of colonies contained only Schwann cells (S only). While Schwann cells in colonies containing neurons always expressed GFAP, colonies that did not include neurons sometimes did not express detectable GFAP but were morphologically indistinguishable from GFAP expressing cells and always expressed p75 and cytoplasmic S100β, but not peripherin or SMA. The frequency of S-only progenitors increased significantly with development, from 20% of colonies at E14.5 to 42% of all colonies at E17.5 (TABLE 1). In standard culture conditions, these colonies typically contained hundreds to thousands of cells. At all stages of development, around 50% of colonies contained only myofibroblasts (M-only). Myofibroblast-only colonies sometimes contained fewer than 10 cells, but in other cases contained more than a hundred cells.

### EXAMPLE 2

### SCIATIC NERVE MULTIPOTENT PROGENITORS SELF-RENEW IN CULTURE

In vitro self-renewal assay. Self-renewal was assayed in vitro by subcloning multipotent progenitors. Single p75⁺ cells from E14.5 sciatic nerve were sorted by FACS into individual wells. of 96 well plates and cultured for 7 to 11 days in standard medium without refeeding. After 7 and 11 days, the wells were examined and multipotent colonies were identified by their appearance. Neural progenitors yielded dense colonies of small cells, while myofibroblast progenitors gave rise to diffuse colonies of large flat fibroblastic cells. Multipotent progenitors could be distinguished from other neural progenitors because they gave rise to larger colonies than S-only or S+M progenitors even by 7 days of culture. Colonies were subcloned by aspirating the culture medium, and adding trypsin-EDTA solution (Gibco) to the well. After 2 min and gentle trituration the cells from individual wells were transferred to a 15 mL tube in which the trypsin was quenched by staining medium with chick embryo extract added. The cells were spun down, resuspended in staining medium, replated in multiple 6-well plate wells at clonal density, and cultured under standard conditions. After 14 days, the composition of the secondary colonies was analyzed immunohistochemically.

Results. The colonies formed by the multipotent progenitors from the sciatic nerve were reminiscent of those formed by migrating NCSCs (see, Anderson et al., U.S. Pat. Nos. 5,589,376, 5,824,489, 5,654,183, 5,693,482, 5,672,499, and 5,849,553, all incorporated by reference; Stemple & Anderson, 71 Cell 973-985 (1992); Shah et al., 77 Cell 349-360 (1994); Shah et al., 85 Cell 331-343 (1996)). The self-renewal potential of the sciatic nerve multipotent progenitors in subcloning tests is presented in TABLE 2.

TABLE 2
Subcloning of multipotent colonies from E14.5 sciatic nerve
after 7 or 11 days in culture

| | |
|---|---|
| Day of | Average number of subclones per founder colony |

| subcloning | N + S + M | N + S | S + M | S only | M only |
|---|---|---|---|---|---|
| 7 | 131 ± 57 | 0.6 ± 1 | 12 ± 13 | 10 ± 7 | 2 ± 2 |
| 11 | 133 ± 165 | 14 ± 14 | 100 ± 45 | 219 ± 138 | 56 ± 58 |
| N, S, and M indicate the presence of neurons, Schwann cells, and myofibroblasts respectively in subcloned colonies. 10 colonies were subcloned at day 7. All colonies yielded N + S + M, S + M, and S-only subclones. Three of 10 colonies also yielded N + S subclones, and 5 of 10 colonies yielded M-only subclones. 10 colonies were subcloned at day 11. 7 of 10 colonies gave rise | | | | | |
| # to at least 10 subclones of each type. 1 colony gave rise to subclones of all types except N + S. Finally 2 colonies gace rise to subclones containing only S and/or M cells, and may have been misidentified as multipotent progenitors. | | | | | |

In 18 out of 20 cases, each multipotent colony gave rise to many multipotent (N+S+M) subclones as well as to S+M subclones and S-only subclones (TABLE 2). In most cases, multipotent colonies also gave rise to M-only and N+S subclones as well. On average, each multipotent founder gave rise to more than 100 multipotent secondary clones irrespective of the day of cloning, corresponding to a minimum of six to seven symmetric self-renewing divisions. Multipotent colonies plated at clonal density from E16.5 sciatic nerves were also subcloned by isolating the colonies with cloning rings. These colonies also self-renewed in culture. Thus the multipotent progenitors not only self-renewed in culture, but gave rise to all other classes of progenitors that were observed in fetal sciatic nerve, including the M-only myofibroblast progenitors. Myofibroblast-only secondary colonies derived from multipotent progenitors (TABLE 2) were phenotypically indistinguishable from those observed in cultures of freshly dissociated sciatic nerve cells (TABLE 1).

Analysis. Thus, single p75⁺P₀⁻ cells have a self-renewal potential that is exhibited in culture.

### EXAMPLE 3

### SEPARATION OF FUNCTIONALLY DISTINCT SCIATIC NERVE PROGENITORS BY FLOW-CYTOMETRY

Introduction. Cells of rat E14.5 sciatic nerve were fractionated by flow-cytometry, using antibodies against cell-surface antigens. Cells dissociated from the sciatic nerve were fractionated into five distinct subpopulations based on differences in their expression of p75, the low-affinity neurotrophin receptor, a marker of neural crest stem cells (NCSCs) (see, Stemple & Anderson, 71 Cell 973-985 (1992)) and P₀, a peripheral myelin protein that has been associated with glial differentiation. Surprisingly, more than 15% of E14.5 sciatic nerve cells were functionally indistinguishable from NCSCs in vitro.

The individual isolated cells self-renewed and gave rise to clones containing neurons, glia, and smooth muscle-like myofibroblasts. These self-renewing multipotent cells were highly enriched in the p75⁺P₀⁻subfraction. These self-renewing multipotent cells responded to instructive lineage determination factors such as BMP2 and neuregulin-1 (NRG-1, also known as glial growth factor) in a manner indistinguishable from NCSCs (see, Shah et al., 77 Cell 349-360 (1994); Shah et al., 85 Cell 331-343 (1996)). Thus, the p75⁺P₀⁻surface marker phenotype permitted the prospective identification and isolation of post-migratory NCSCs from the E14.5 sciatic nerve.

Flow-cytometry procedure. All sorts and analyses were performed on a FACSVantage dual-laser flow-cytometer (Becton-Dickinson, San Jose). In order to isolate NCSCs, E14.5 sciatic nerve cells were stained with antibodies against p75 and P₀. Dissociated sciatic nerve cells were first suspended in a 1/2000 dilution of P07 monoclonal antibody against P₀ (J. J. Archelos, Munich). At higher concentrations P07 tends to bind non-specifically, but at this dilution P07 did not stain fetal liver or dissociated telencephalon cells. All antibody incubations were carried out for 20-25 min on ice. At the end of the incubation period, the cells were washed by diluting in 10 to 40 volumes of staining medium, pelleting the cells by centrifuging for 3 min at 450×g, and then aspirating the staining medium. P07 staining was developed by incubating in an anti-mouse IgG₁ second stage antibody conjugated to phycoerythrin (Southern Biotechnology Associates, Birmingham Ala.). There was no background staining from this second stage antibody on sciatic nerve, fetal liver or telencephalon cells. After washing, the cells were resuspended in 192 IgG antibody (against p75) directly conjugated to fluorescein. 0.1 mg/mL mouse IgGI (Sigma) was included with 192 IgG to block binding to second stage antibody on the cell surface. After washing, the cells were resuspended in staining medium containing 2 μg/mL 7-aminoactinomycin D (7-MD, Molecular Probes, Eugene), a viability dye. Dead cells were excluded by gating on forward and side scatter as well as by eliminating 7-MD positive events. Sorts were performed using the Clone-Cyte function to deposit known cell numbers directly into individual wells of culture plates. In order to calculate plating efficiencies, the accuracy of Clone-Cyte sort counts was checked regularly by sorting cells onto glass slides and counting the number of cells that were actually sorted. Prior to and after sorts, tissue culture plates were kept in sealed plastic bags gassed with 5% CO₂ to prevent the culture medium pH from becoming basic by equilibrating with the air.

Cell cycle analyses of NCSCs were performed by staining with Hoechst 33342 (Sigma) to measure DNA content, and pyronin Y (Sigma) to measure RNA content. At least 5500 p75⁺P₀⁻cells from E14.5 sciatic nerve were sorted into staining medium and then pipetted into ice cold 70% ethanol. The cells were left in ethanol at 4° C. overnight, then resuspended in 1 μg/mL Hoechst 33342 plus 2 μg/mL pyronin Y 20 min before flow-cytometric reanalysis. At least 1500 p75⁺P₀⁻cells were reanalyzed per replicate. Instrument parameters for analyzing DNA and RNA content were set based on quiescent (adult rat splenocytes) and actively dividing (rat telencephalon) control cells.

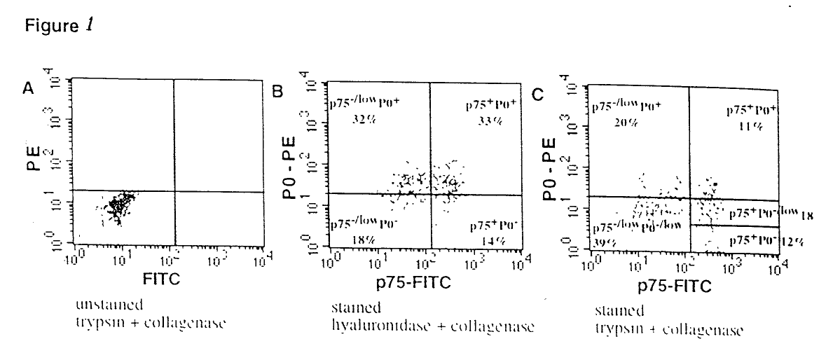

Results. FIG. 1 shows FACS plots of dissociated E14.5 sciatic nerve cells, either unstained, or stained with p75 and P₀. For phenotypic analyses, we used two different dissociation conditions: (1) hyaluronidase+collagenase, which minimized protease activity and thus favored the retention of cell surface markers, or (2) trypsin+collagenase, which favored cell survival and high plating efficiencies. By comparing hyaluronidase+collagenase dissociated cells doubly labeled with anti-p75 and anti-P₀ with unstained cells, four phenotypically distinct populations of sciatic nerve cells were defined according to their expression of p75 and P₀: (1) p75⁺P₀⁺, (2) p75⁺P₀⁺, (3) p75⁻/^{low}P₀⁺, and (4) p75^{-/low}P₀⁻(FIG. 1A, B). As expected, most (65%) E14.5 sciatic nerve cells expressed P₀, and many (47%) cells expressed p75, following hyaluronidase+collagenase digestion. Neither of these antibodies exhibited non-specific staining at the concentrations used to stain sciatic nerve cells when tested by FACS on telencephalon or fetal liver control cells.

Unfortunately, a relatively low proportion of hyaluronidase+collagenase dissociated cells formed colonies in culture, hampering our ability to compare the developmental potentials of these populations. However, cells dissociated by a brief (4 min) trypsin+collagenase treatment did efficiently form colonies; therefore, all functional analyses were performed on such trypsin+collagenase dissociated cells. The FACS plots showed a slight loss of P₀ epitopes, but no loss of p75 epitopes by this dissociation procedure (compare FIG. 1B and 1C). Because of this dimming of P₀ staining, we divided trypsin+collagenase dissociated cells into five subsets (FIG. 1C). p75⁺P₀⁻cells represented 12±2% of sciatic nerve cells, p75⁺P₀⁻represented 12±2% of sciatic nerve cells, p75⁺P₀^{-/low} cells represented 18±5% of sciatic nerve cells, p75⁺P₀⁺represented 11±7&, p75^{-/low}P₀⁺cells represented 20±9%, and p75^{-/low}P₀^{-/low} low cells represented 39±10%.

Each of the 5 populations from the trypsin+collagenase dissociated sciatic nerve was cultured under standard conditions. The populations showed striking differences in developmental potential, as shown in TABLE 3.

TABLE 3
Developmental potentials of phenotypically distinct populations
from the El4.5 sciatic nerve in clonal culture

| | Plating | Frequency of colony types (% ± std. dev) | | | | |
|---|---|---|---|---|---|---|
| Population | efficiency | N + S + M | N + S | S + M | S only | M only |
| p75⁺ P₀⁻ | 24.9 ± 8.8 | 60.4 ± 3.5^{a} | 4.8 ± 8.2 | 11.6 ± 16.1 | 18.4 ± 7.4^{a} | 4.8 ± 8.3^{a} |
| p75⁺ P₀^{-/low} | 36.8 ± 6.3 | 50.4 ± 11.9^{a} | 0.0 | 5.7 ± 6.9 | 371 ± 12.8^{b} | 6.8 ± 8.8^{a} |
| p75⁺ P₀⁺ | 48.2 ± 16.4 | 27.8 ± 12.8^{b} | 4.5 ± 6.5 | 11.7 ± 6.0 | 34.2 ± 15.8^{a,b} | 21.8 ± 11.0^{b} |
| p75^{-/low} P₀⁺ | 84.7 ± 17.5 | 0.0^{c} | 0.0 | 0.4 ± 0.8 | 0.4 ± 0.8^{c} | 99.2 ± 1.6^{c} |
| p75^{-/low} P_{0 -} | 52.4 ± 9.9 | 0.0^{c} | 0.0 | 0.0 | 0.0^{c} | 100.0 ± 0.0^{c} |

Statistics within columns of colony-type data were compared by analyses of variance followed by post-hoc T-tests. Columns containing significantly different statistics (p < 0.001 by anova) include letters to designate the pair-wise differences. Significantly different statistics are not followed by the same letter (e.g., a is different from b but not from a,b).

Most p75⁺P₀⁻cells formed multipotent colonies (60%), with smaller numbers of cells giving rise to the other classes of colonies. p75⁺P₀⁻cells gave rise to a mixture of multipotent colonies (50%) and Schwann-only colonies (37%). Both of these fractions gave rise to a low percentage of M-only colonies (<10%). The p75⁺P₀⁻population contained a mixture of multipotent progenitors (28%), Schwann only progenitors (34%), and myofibroblast-only progenitors (22%). Thus all p75⁺ populations, including those expressing P₀, contained substantial numbers of multipotent progenitors, but the apparent frequency of multipotent progenitors among cells that -how formed colonies decreased. as P₀ expression increased. Both of the p75 flow populations (P₀⁺ and P₀) were pure, or nearly pure populations of progenitors that gave rise only to myofibroblasts.

p75⁺P₀⁻E14.5 sciatic nerve cells are enriched in NCSCs. Although the p75⁺P₀⁻population was enriched for NCSCs, it was not pure. Sixty percent of cells in standard culture formed self-renewing multipotent colonies. Over 80% of cells were capable of generating neurons in the presence of BMP2. No committed neuronal progenitors were detected in this or previous studies of the sciatic nerve, and in the absence of BMP2 neuronal progenitor activity was always associated with multipotent progenitors (TABLES 1 and 3); therefore, the results in the presence of BMP2 suggest that some of the non-neurogenic colony types that formed under standard conditions may have been NCSCs that failed to “read out” their neuronal potential at the time point assayed. Thus, up to 80% of p75⁺P₀⁻cells that formed colonies under our culture conditions may be NCSCs. Since only 15-16% of unfractionated sciatic nerve cells behaved as NCSCs (TABLE 1), the p75⁺P₀⁻fraction is enriched for stem cells approximately 4 to 5-fold.

### EXAMPLE 4

### NEURONAL POTENTIALS DETERMINED BY BMP2 CHALLENGE

BMP2 instructs NCSCs to differentiate into neurons. 1.6 nM BMP2 was therefore added to standard cultures of unseparated sciatic nerve cells or cells from each subpopulation isolated by flow-cytometry. After 24 hr with BMP2 some cultures were fixed and stained for MASH-1, an early transcription factor marker of autonomic neurogenesis. After 4 days with BMP2 sister cultures were fixed and stained for peripherin, a marker of mature PNS neurons. On average, 18-20% of unseparated sciatic nerve cells were capable of neuronal differentiation, as judged by either MASH-1 or peripherin expression (TABLE 4).

TABLE 4
Neuronal potentials of phenotypically distinct
populations from the E14.5 sciatic nerve
challenged by BMP2 in clonal culture.

| | |
|---|---|
| 24 hr challenge | 4 day challenge |
| (% MASH-l⁺) | (% peripherin⁺) |

| Population | no add | +BMP2 | no add | +BMP2 |
|---|---|---|---|---|
| unseparated cells | 0.0 | 19.6 ± 14.5^{a} | 0.0 | 18.0 ± 13.6^{a} |
| p75⁺ P₀⁻ | 0.0 | 81.0 ± 11.5^{b} | 0.0 | 82.2 ± 10.9^{b} |
| p75⁺ P₀^{-/low} | 0.0 | 68.0 ± 9.4^{b} | 0.0 | 68.1 ± 18.8^{b} |
| p75⁺ P₀⁺ | 0.0 | 42.5 ± 20.7^{c} | 0.0 | 52.9 ± 14.6^{c} |
| p75^{-/low} P₀⁺ | 0.0 | 0.7 ± 1.2^{d} | 0.0 | 0.9 ± 1.4^{d} |
| p75^{-/low} P₀⁻ | 0.0 | 1.1 ± 2.1^{d} | 0.0 | 3.8 ± 3.6^{e} |
| Cells from each population were sorted into culture with or without BMP2. After 24 hr, some cultures were fixed and stained for MASH-1, a marker of neuronal differentiation, while other cultures were fixed at 4 days and stained for peripherin, a marker of mature neurons. BMP2 addition did not significantly affect the plating efficiency of any population at either time point. Statistics within | | | | |
| # columns were compared by analyses of variance followed by post-hoc T-tests. Columns containing significantly different statistics (p < 0.001 by anova) include letters to designate the pair-wise differences. Significantly different statistics are not followed by the same letter (e.g., a is different from b or c). | | | | |

These results are consistent with our observation that 16% of unseparated sciatic nerve cells formed colonies that contained neurons in standard cultures (TABLE 1).

In the p75⁺P₀⁻population, in which 60% of cells gave rise to multipotent progenitors in standard culture (TABLE 3), over 80% of the cells differentiated into neurons in the presence of BMP2 (TABLE 4). Thus in the presence of BMP2 a higher proportion of cells exhibited neurogenic potential. In the p75⁺P₀⁻ and p75⁺P₀⁺populations, substantial but lower numbers of cells (68% and 52% respectively) differentiated into neurons under the influence of BMP2. This is consistent with the observation that many cells in these populations were multipotent progenitors, capable of neuronal differentiation (TABLE 3). By contrast, in both of the p75 populations (P₀⁺ and P₀⁻) few cells were capable of neuronal differentiation, even when challenged by BMP2 (TABLES 3 and 4). This demonstrates that most cells in these populations lack neuronal potential and is consistent with the possibility that almost all cells in these populations are restricted to myofibroblast fates.

BMP2 did not appear to either kill cells or promote the survival of subpopulations of cells because in no case was there a difference in plating efficiency comparing side-by-side cultures with and without BMP2, either after 24 hr or after 4 days. In the absence of BMP2, peripherin staining was not apparent until day 13 under standard culture conditions, but in the presence of BMP2, neuronal differentiation occurred within 4 days. In the absence of BMP2 only a minority of cells in p75⁺P₀⁻colonies were neurons. In the presence of BMP2 all cells in most p75⁺P₀colonies were neurons. Since BMP2 accelerated neuronal differentiation and dramatically increased the proportion of neurons in clones, but did not appear to affect cell survival, the data suggest that it acted instructively on cells with neuronal potential.

To confirm that BMP2 was acting instructively, p75⁺P₎⁻cells from the E14.5 sciatic nerve were sorted into culture. After 4 hr the cultures were examined microscopically, and live cells that had attached to the plate were marked by etching a circle on the underside of the culture plate. After cells were circled, BMP2 was added to some cultures. 24 hr after BMP2 addition, cultures were fixed and stained for MASH-1. In cultures that did not contain BMP2, an average of 88.9% of cells survived and no cells expressed MASH-1 (45 founder cells studied in 2 tests). In cultures to which BMP2 was added, an average of 87.5% of cells survived and 62.7% of those cells expressed MASH-1 (40 founder cells studied in 2 tests). Thus BMP2 did not act selectively, but instructed sciatic nerve multipotent progenitors to differentiate into the neuronal lineage, similar to its effect on NCSCs obtained from E10.5 neural tube explants, as shown by Shah et al., 85 Cell 331-343 (1996)).

### EXAMPLE 5

### GLIAL POTENTIALS DETERMINED BY NRG-1 CHALLENGE

NRG-1 instructs migrating NCSCs to differentiate into glia. Cultures of each population, isolated by FACS from E14.5 sciatic nerve, were challenged by adding 1 nM NRG-1. After 14 days, the cultures were fixed and stained for peripherin, GFAP, and SMA. The results are presented in TABLE 5.

TABLE 5
Glial potentials of phenotypically distinct populations from the E14.5 sciatic nerve as
determined by challenge with NRG-l (glial growth factor) in clonal culture.

| | Plating | Frequency of colony types (% ± std. dev) | | | |
|---|---|---|---|---|---|
| Population | efficiency | N + S + M | S + M | S only | M only |
| unseparated | 67.1 ± 0.8 | 3.3 ± 5.8 | 11.6 ± 5.7^{a,d} | 38.5 ± 31.6^{a} | 46.6 ± 31.6^{a} |
| p75⁺ P₀⁻ | 55.2 ± 3.8 | 0.0 | 5.0 ± 3.3^{a} | 95.0 ± 3.3^{b} | 0.0^{b} |
| p75⁺ P₀^{-/low} | 54.4 ± 10.7 | 0.7 ± 1.4 | 20.9 ± 21.0^{a,b,c} | 79.1 ± 21.0^{b,e} | 0.0^{b} |
| p75⁺ P₀⁺ | 64.8 ± 15.4 | 2.0 ± 3.1 | 12.0 ± 5.8^{b,d} | 84.4 ± 7.3^{c,e} | 1.6 ± 24^{b} |
| p75^{-/low} P₀⁺ | 68.9 ± 17.5 | 0.0 | 0.0^{c} | 0.0^{d} | 100.0 ± 00^{c} |
| p75^{-/low} P₀⁻ | 41.5 ± 20.4 | 0.0 | 0.0^{c} | 0.0^{d} | 100.0 ± 00^{c} |
| Cells from each population were sorted into cultures containing NRG-1. After 2 weeks, the cultures were fixed and stained. No colonies containing only neurons and Schwann cells (N + S) were observed in these tests. Statistics within columns of colony-type data were compared by analyses of variance followed by post-hoc T-tests. Columns containing significantly different statistics | | | | | |
| # (p < 0.05 by anova) include letters to designate the pair-wise differences. Significantly different statistics are not followed by the same letter (e.g., a is different from b but not from a,b). | | | | | |

In contrast to the neuronal differentiation seen in cultures of p75⁺P₀⁻cells under standard conditions (TABLE 3), in the presence of NRG-1 no neuron-containing colonies were observed (TABLE 5, N+S+M) and 95% of colonies contained only Schwann cells. Indeed, neuronal differentiation was suppressed in the presence of NRG-1 in cultures of both unseparated cells and p75⁺ populations, while the frequencies of colonies containing only Schwann cells were dramatically increased (compare S-only values in TABLES 1, 3, and 5 - note that the data in these TABLES were obtained in side-by-side cultures in the same tests). Plating efficiencies were also significantly higher for p75⁺ populations in the presence of NRG- 1 (compare plating efficiencies in TABLES 3 and 5). Thus NRG-1 also acted as a survival factor for neural progenitors as previously reported (Dong et al., 1995). Neither the plating efficiency nor the differentiation of p75 progenitors were affected by NRG-1 challenge. 100% of colonies from p75 progenitors contained only myofibroblasts expressing SMA, even in the presence of NRG-1 (TABLE 5). Thus these myofibroblast progenitors appear to have neither glial nor neuronal potential. NRG-1 did, however, promote the proliferation of cells in colonies derived from p75 progenitors.

To confirm that NRG-1 also acted instructively to promote glial differentiation by multipotent progenitors from the E14.5 sciatic nerve, we plated p75⁺P₀⁻cells in the absence of NRG-1. Four hr after plating, live p75⁺P₀ cells were circled and then NRG-1 was added to the cultures. After 14 days the cultures were fixed and stained. In the presence of NRG-1 an average of 95.2% of colonies survived and all of these cells gave rise to glial containing colonies as judged by morphology, and p75 or S100β staining (60 colonies examined in three tests). Thus NRG-1 promoted glial differentiation without killing multipotent progenitors, demonstrating that it acted instructively. In addition to this instructive effect, the increase in plating efficiency noted above suggests that it may also promote the survival of p75⁺ progenitors.

The foregoing data suggested that the multipotent progenitors in the E14.5 sciatic nerve were phenotypically and functionally indistinguishable from migrating NCSCs isolated from E10.5 neural tube explants, by the following criteria: (1) both express p75 (Stemple & Anderson, 71 Cell 973-985 (1992)); (2) both generate multipotent colonies containing neurons, glia, and SMA⁺ myofibroblasts, and self-renew in culture (Stemple & Anderson, 71 Cell 973-985 (1992)); Shah et al., 85 Cell 331-343 (1996)); (3) both are instructed by BMP2 to differentiate into neurons as evidenced by expression of MASH-1 within 24 hr and peripherin within 4 days; and (4) both are instructed by NRG-1 to differentiate into glia. The only other published functional characteristic of migrating NCSCs is that TGFβ instructs them to differentiate into SMA⁺ calponin⁺ cells that were described as smooth muscle cells by Shah et al., 85 Cell 331-343 (1996), but which are morphologically and antigenically indistinguishable from the cells described here as myofibroblasts. NCSCs replated from neural tube explants and p75⁺P₀⁻cells from the E14.5 sciatic nerve responded indistinguishably to TGFβ challenge under standard culture conditions. For reasons that are not clear, in the present tests, a higher proportion of cells failed to survive in TGFβ than was observed in by Shah et al., 85 Cell 331-343 (1996), but surviving cells were enriched for SMA⁺myofibroblasts, consistent with an instructive role for TGFβ. Thus, the multipotent progenitors observed in the fetal sciatic nerve are NCSCs.

### EXAMPLE 6

### p75⁺P₀⁻NCSCs FROM THE E14.5 SCIATIC NERVE GIVE RISE TO NEURONS AND GLIA UPON TRANSPLANTATION IN VIVO

Introduction. To determine whether freshly isolated sciatic nerve p75⁺P₀⁻cells were multipotent in vivo, and to ensure that their neuronal potential was not acquired by dedifferentiation in vitro, we used a system for transplantation of rat neural crest cells into chick embryos. p75⁺P₀ cells from freshly dissociated sciatic nerves were isolated by FACS and injected into somites at either the forelimb or sacral level of stage 18 chick embryos. Thus the donor cells were placed in the ventral neural crest pathway, at a developmental stage when host crest migration is well underway.

In vivo transplantation of sciatic nerve progenitors. Fertile white Leghorn eggs were incubated to Hamburger and Hamilton stage 18. 20,000 to 90,000 p75⁺P₀⁻cells from E14.5 sciatic nerve were isolated by FACS, added to a drawn glass capillary tube and allowed to sediment by gravity toward the tip at 4° C. for 30 min. The injection process was performed as described by Bronner-Fraser et al., 77 Developmental Biology 130-141 (1980). A small bolus of 10% India ink in calcium and magnesium free Tyrode's salt solution was injected under the blastoderm to visualize the embryo. The cells were injected into the anterior, medial corner of one or two somites of each embryo, using an MM33 micromanipulator (Fine Science Tools) and very gentle air pressure. Numerous embryos were injected with each cell preparation. In similar control tests done with rat neural crest outgrowth, a number of injected embryos were immediately fixed and the number of injected cells were counted. Although variation is inherent in the method 200 to 600 cells were consistently observed to localize within the embryo. Injected embryos were incubated for an additional 3 days, to stage 29. Embryos were then fixed by immersion in fresh, ice cold, 4% paraformaldehyde in phosphate buffer for at least 16 hr, sunk in 15% sucrose, embedded in OCT, and stored frozen at −80° C. Fifteen μm sections were cut of selected portions or the embryos. Normal rat and chick embryos were processed in parallel as positive and negative controls for in situ hybridization.

In situ hybridization procedure. Three days after injection the chick embryos were harvested, sectioned, and stained by in situ hybridization with rat and chick-specific probes against markers of neurons and glia. We have determined that three days of incubation is sufficient for the rat donor cells to migrate to normal crest locations and to begin the process of differentiation.

Antisense probes for rat-specific genes were synthesized with digoxygenin-conjugated nucleotides, and antisense probes for chick-specific genes were synthesized with fluorescein-conjugated nucleotides. Detailed protocols are available upon request. Briefly, sections were post-fixed, digested with proteinase K, and acetylated. Samples were then pre-hybridized for 1 to 3 hr at 65° C. and hybridized with 1 mg/ml probe overnight at 65° C. Three high-stringency washes with 0.2×SSC were done at 65° C. Blocking was done with 20% sheep serum for an hour at room temperature, and slides were incubated with pre-absorbed alkaline phosphatase-conjugated anti-digoxygenin antibody in blocking solution for one hr at room temperature. Slides were developed with Nitro blue tetrazolium (NBT) and 5-bromo-4-chloro-3-indoly phosphate (BCIP). After NBT/BCI P development, the digoxygen in-conjugated alkaline phosphatase was inactivated by heating to 85° C.; the slides were incubated in alkaline phosphatase-conjugated anti-fluorescein antibody, and developed with 2-[4-iodophenyl]-3-[4-nitrophenyl]-5-phenyl tetrazolium chloride (INT) and BCIP, which yields an orange product. This combination accurately distinguished between graft and host, because chicken probes effectively prevented cross-hybridization of rat probes in negative control chick embryos and in the CNS motor neuron pools of injected embryos. Since rat cells were only injected on one side of each embryo, the contralateral side of the same embryos served as an additional internal control for the specificity of staining.

Engraftment results. E14.5 p75⁺P₀⁻donor cells engrafted efficiently and gave rise to neurons and glia in diverse PNS locations. In two tests, such cells were injected into a total of 22 chick embryos. Engraftment of rat cells occurred in 16 of 18 forelimb injected chick embryos and 4 of 4 sacrally injected chick embryos, generating a total of 20 chimeras. Donor derived neurons, identified by in situ hybridization with a rat-specific probe for the neuronal marker SCG 10, were detected in the sympathetic ganglia of 4 chimeras (3 forelimb and 1 sacral injection) in close association with host neurons counter-stained with a chick-specific SCG10 probe (orange stain). Rat cells that engrafted in sympathetic ganglia also expressed Phox2b, which is a marker of autonomic differentiation appropriate to the sympathetic ganglion. Among chicks injected at sacral levels, rat neurons were always detected in Remak's ganglion, a component of the avian enteric nervous system.

In addition to the engraftment of neurons, cells expressing P₀ and the NRG-1 receptor erbB3 were detected in the peripheral nerves of all chimeras, sometimes numbering into the hundreds. These cells did not express SCG10 in adjacent sections.

Analysis. Thus some engrafting rat cells differentiated appropriately in peripheral nerves by forming Schwann cells and not neurons. Taken together, the results demonstrate that sciatic nerve p75⁺P₀⁻cells can give rise to neurons as well as glia in vivo, when transplanted directly after flow cytometric isolation without any intervening. period of growth in culture.

### EXAMPLE 7

### NCSCs PERSIST BY SELF-RENEWING IN THE SCIATIC NERVE

Introduction. The persistence of NCSCs in the fetal sciatic nerve could reflect their survival in a mitotically quiescent state following immigration from the neural crest. Alternatively, the cells could persist by undergoing self-renewing divisions.

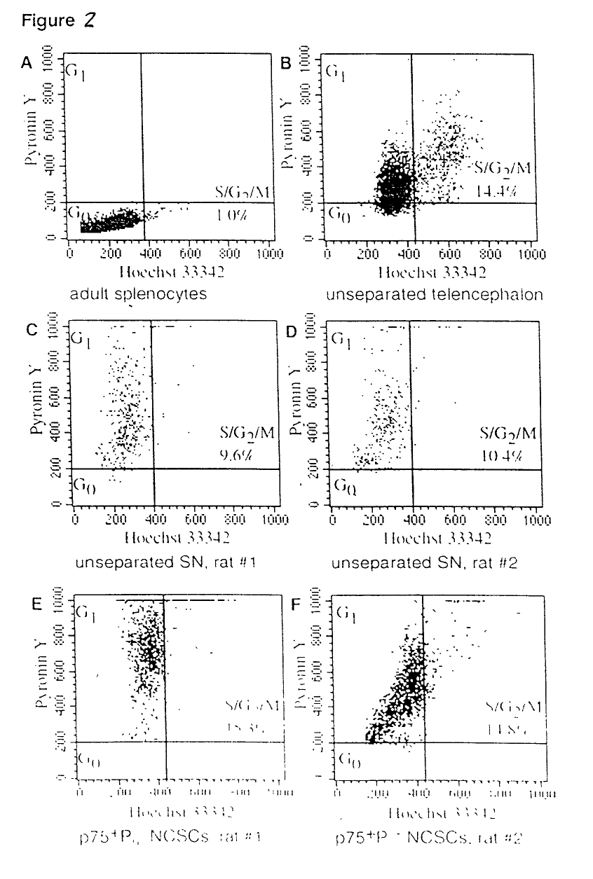

To distinguish these possibilities, we first examined the cell cycle status of p75⁺P₀⁻cells from the E14.5 sciatic nerve. p75⁺P₀⁻cells were isolated by FACS, then stained with Hoechst 33342 and pyronin Y and reanalyzed by FACS to determine their DNA and RNA contents. Using this approach, cells can be assigned to G₀, G₁, or SG₂/M phases of the cell cycle. Both unfractionated E14.5 sciatic nerve cells (FIG. 2C, 2D) and p75⁺P₀⁻cells (FIG. 2E, 2F) appeared to be rapidly cycling populations with many cells in S/G₂/M and few or no cells in G₁. About 10% of unfractionated sciatic nerve cells were in S/G₂/M, while about 15% of p75⁺P₀⁻cells from the same nerves were in S/G₂/M.

To directly assay whether most NCSCs were self-renewing in vivo, pregnant rats were administered the thymidine analogue BrdU for 18 hr prior to the harvest of fetal sciatic nerves at E14.5 (i.e., at E13.75).

Self-renewal assays. Self-renewal was assayed in vivo by administering 5′-bromo-2′-deoxyuridine (BrdU, Sigma) to pregnant rats for 18 hr prior to harvest of sciatic nerves from pups at E14.5. Doses of BrdU equivalent to 50 pg/g body weight were dissolved in 1 mL D-PBS with 0.007 M NaOH and injected i.p. at harvest −18 hr, −16hr, −14hr, −4hr, and −2hr. Additionally, at harvest −14 hr, the rat's normal water was replaced by water containing 2 mg/mL BrdU. After dissecting sciatic nerves, the cells were stained as described above and p75⁺P₀⁻cells were sorted into culture. After letting the cells adhere to the culture dish for 3-4 hr under standard culture conditions, the cells were stained with an antibody against BrdU.

Results. Unfractionated sciatic nerve cells, and p75⁺P₀ cells isolated by FACS were plated, fixed and stained for BrdU incorporation. 80% of sciatic nerve cells, and nearly 90% of p75⁺P₀⁻cells, incorporated BrdU over the 18 hr pulse in vivo (TABLE 6).

TABLE 6
Cumulative BrdU labeling of neural crest cells in the sciatic nerve
by continuous administration of BrdU for 18 hr prior to harvest at E14.5.

| | | % BrdU⁺ |
|---|---|---|
| Unseparated SN | BrdU treated in vivo | 79.8 ± 7.6 |
| | normal | 0.0 |
| | normal, 6h BrdU in vitro* | 18.9 ± 7.3 |
| p75⁺ P₀⁻ | BrdU treated in vivo | 89.3 ± 6.0 |
| | normal | 0.0 |

*normal, freshly dissociated sciatic nerve cells were cultured under standard conditions for 5 to 7 hr with NRG-1 and 10 μM BrdU added.

We confirmed in several ways that BrdU administration did not disrupt normal development within the sciatic-nerve. Unseparated sciatic nerve cells from BrdU administered and normal rats were indistinguishable in terms of their FACS profiles and their expression of p75 and P₀. Unseparated and p75⁺P₀⁻cells from the sciatic nerves of normal (FIG. 2D, 2F) and BrdU administered (FIG. 2E) rats also did not differ in terms of cell cycle status. Unseparated cells from BrdU administered rats also did not differ significantly from normal rats in terms of the number of cells that expressed MASH-1 after a 24 hr BMP2 challenge, or the number of N+S+M colonies that they formed after a two week culture.

Analysis. Earlier retroviral lineage marking experiments in the fetal CNS and retina provided evidence for proliferation within clones containing neurons and glia. However, the lack of markers to distinguish stem cells from committed progenitors made it impossible to test whether such proliferation reflected the self-renewal of multipotent progenitors or the expansion of restricted progenitors. Indeed it has been noted that retroviral marking does not indicate developmental potential and therefore cannot definitively identify stem cells (Turner et al., 4 Neuron 833-845 (1990)).

The NCSCs that we identified in E14.5-E17.5 sciatic nerves derive from neural crest cells that had migrated there several days earlier. The persistence of these multipotent cells could reflect their self-renewal in the nerve. Alternatively, these cells could persist in a quiescent state in vivo, from which they could be induced to re-enter the cell cycle upon culturing in vitro. 90% of p75⁺P₀⁻cells were labeled in vivo by an 18 hour (hr) pulse of BrdU administered from E13.75 to E14.5. Therefore, these cells were undergoing active divisions prior to isolation at E14.5. Furthermore, they must have incorporated BrdU after their arrival in the nerve, rather than prior to emigration from the neural tube, since the migration of trunk neural crest cells along the ventro-lateral pathway in the rat is likely over by E11.5-E12.0. Although late-emigrating cells from the dorsal spinal cord in chick differentiate to neurons and satellite cells in the dorsal root ganglia, no evidence was seen that individual cells were multipotent, or that they contributed to the peripheral nerve. Thus, by the time of BrdU administration at E13.75, neural crest-derived cells were likely already resident in the peripheral nerve for several days. Cells that incorporated BrdU in vivo retained their capacity to generate neurons, Schwann cells and myofibroblasts when subsequently isolated; furthermore, these cells were functionally identical to NCSCs isolated from 24 hr explants of E10.5 neural tubes.

Thus, the divisions that these cells underwent in vivo must have been self-renewing.

These data are consistent with the cell cycle analysis, in demonstrating that the p75⁺P₀⁻cells were dividing rapidly. We confirmed that the p75⁺P₀⁻population from BrdU administered rats remained enriched for NCSCs by observing that 86% of such cells expressed MASH-1 after a 24 hr BMP2 challenge, and that in standard culture conditions an average of 50% of the colonies formed by such cells contained neurons, Schwann cells and myofibroblasts (N+S+M). Since 90% of p75⁺P₀⁻cells were BrdU⁺, these data indicate that most or all cells that retained multilineage differentiation activity after isolation had previously incorporated BrdU in vivo. These data indicate that NCSCs undergo self-renewing divisions in vivo.

The frequency of NCSCs within the p75⁺P₀⁻population may have been, if anything, underestimated, because NCSCs may form colonies less efficiently than the restricted progenitors that were also observed in the p75⁺P₀⁻population. While the p75⁺P₀⁻population may be only 60-80% pure, this level of purity was more than sufficient to demonstrate self-renewal by BrdU labeling since almost 90% of p75⁺P₀⁻cells incorporated BrdU (TABLE 6). In addition, this calculation of 90% BrdU cells is based on the cells that plated under standard culture conditions, so it is particularly unlikely that cells incapable of surviving in standard cultures could have skewed the BrdU analysis.

The details of one or more embodiments of the invention are set forth in the accompanying description above, though any methods and materials similar or equivalent to those described herein can be used in the practice or testing of the present invention, the preferred methods and materials are described. Other features, objects, and advantages of the invention will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this invention belongs. All patents and publications cited in this specification are incorporated by reference.

The foregoing description has been presented only for the purposes of illustration and is not intended to limit the invention to the precise form disclosed, but by the claims appended hereto.

We claim:

1. A method for enriching a population of uncultured cells for neural stem cells, comprising:
(a) contacting a population of uncultured cells containing a neural stem cell with a combination of reagents, wherein each reagent in the combination either selectively binds to a either neural stem cell positive marker or a neural stem cell negative marker; and
(b) selecting cells which bind to reagents that selectively bind to a positive marker or which do not bind to reagents that selectively bind to a negative marker or a combination thereof, wherein the selected cells are enriched in neural stem cells as compared with the population of uncultured cells.

2. The method of claim 1, wherein the neural stem cell is a neural crest stem cell (NCSC).

3. The method of claim 1, wherein the neural stem cell is a central nervous system (CNS) neural stem cell.

4. The method of claim 1, wherein the selected cells are at least 50% neural stem cells.

5. The method of claim 1, wherein a reagent is an antibody.

6. The method of claim 1, wherein a reagent is an anti-p75 (low-affinity neurotrophin receptor) antibody.

7. The method of claim 1, wherein a reagent is an anti-P₀ antibody.

8. The method of claim 1, wherein the population of uncultured cells is derived from the neural crest.

9. The method of claim 1, wherein the population of uncultured cells is dissociated neural tissue.

10. The method of claim 1, wherein the population of uncultured cells is dissociated peripheral nerve.

11. The method of claim 1, wherein the selecting is by flow cytometry.

12. The method of claim 1, further comprising:
(c) transplanting the selected cells into a host.

13. A method for enriching a population of cells for the neural stem cell fraction, comprising:
(a) contacting a population of cells containing a fraction of neural stem cells with a reagent that specifically binds to p75 (low-affinity neurotrophin receptor); and
(b) selecting p75⁺ cells, wherein the selected p75⁺ cells are enriched in the fraction of neural stem cells as compared with the unselected population of cells.

14. The method of claim 13, further comprising:
(c) contacting the selected p75⁺ cells with a reagent that specifically binds to the P₀ antigen; and
(d) selecting P₀⁻cells, wherein the selected p75⁺P₀⁻cells are enriched in the fraction of neural stem cells as compared with the population of neural cells.

15. A method for isolating a neural stem cell, comprising:
(a) contacting a population of uncultured cells containing a neural stem cell with a combination of reagents, wherein each reagent in the combination either selectively binds to either a neural stem cell positive marker or a neural stem cell negative marker;
(b) selecting cells which bind to reagents that selectively bind to a positive marker or which do not bind to reagents that selectively bind to a negative marker or a combination thereof,
(c) introducing at least one selected cell to a culture medium capable of supporting the growth of neural stem cells; and
(d) proliferating the selected cell in the culture medium, wherein the proliferated progeny cells are derived from an isolated neural stem cell.

16. The method of claim 15, wherein the culture medium capable of supporting the growth of neural stem cell comprises a serum free-medium containing chick embryo extract.

17. The method of claim 15, further comprising:
(e) differentiating the proliferated progeny cells to produce a cell culture comprising differentiated cells selected from the group consisting of neurons, glia, myofibroblasts, and combinations thereof.

18. The method of claim 15, further comprising:
(e) transplanting the proliferated progeny cells into a host.

19. The method of claim 15, further comprising:
(e) contacting the proliferated progeny cells with a biological agent; and
(f) determining the effects of the biological agent on the proliferated progeny cells.

20. The method of claim 15, further comprising:
(e) inducing the proliferated progeny cells to differentiate in a second culture medium containing a biological agent; and
(f) determining the effects of the biological agent on the differentiated cells.

21. The method of claim 15, further comprising:
(e) inducing the proliferated progeny cells to differentiate in a second culture containing a biological agent;
(f) contacting the differentiated cells with the biological agent; and
(g) determining the effects of the biological agent on the differentiated neural cells.

22. An in vitro cell culture composition, comprising:
(a) a population comprising at least 50% self-renewing multipotent neural stem cells, wherein the neural stem cells have been derived from uncultured tissue; and
(b) a culture medium that supports the growth of neural stem cells.

23. The composition of claim 22, wherein the population of cells are derived from dissociated nerves.

24. The composition of claim 22, wherein the population of cells are derived from primary peripheral nervous system (PNS) tissue.

25. The composition of claim 22, wherein the population of cells are derived from primary central nervous system (CNS) tissue.

26. The composition of claim 22, wherein the population of cells are derived by immunoselection using an anti-p75 antibody.

27. The composition of claim 22, wherein the population of cells are derived by immunoselection using an anti-P₀ antibody.

28. The composition of claim 22, wherein the population of cells has at least 80% p75⁺ cells.

29. The composition of claim 22, wherein the neural stem cells are rat.

30. The composition of claim 22, wherein the neural stem cells are chick.

31. The composition of claim 22, wherein the neural stem cells are human.

32. The composition of claim 22, wherein the culture medium comprises a serum free-medium containing chick embryo extract.

33. The composition of claim 22, wherein the culture medium comprises an instructive factor.

34. The composition of claim 33, wherein the instructive factor is a growth factor of the TGF-β superfamily.

35. The composition of claim 33, wherein the instructive factor is a neuregulin (NRG-1).
